(19) United States
(12) Patent Application Publication
(10) Pub. No.: US 2022/0041751 A1
(43) Pub. Date: Feb. 10, 2022

(54) COMBINATION THERAPY FOR THE TREATMENT OF CANCER

(71) Applicant: Board of Regents, The University of Texas System, Austin, TX (US)

(72) Inventors: John V. HEYMACH, Houston, TX (US); Jacqulyne ROBICHAUX, Houston, TX (US); Monique NILSSON, Houston, TX (US)

(73) Assignee: Board of Regents, The University of Texas System, Austin, TX (US)

(21) Appl. No.: 17/416,227

(22) PCT Filed: Dec. 20, 2019
(86) PCT No.: PCT/US2019/068153

§ 371 Date: Jun. 18, 2021

Related U.S. Application Data
(60) Provisional application No. 62/784,084, filed on Dec. 21, 2018.

(51) Int. Cl. C07K 16/32; A61K 31/517; A61K 45/06; A61K 47/68; A61P 35/00

(52) U.S. Cl. CPC C07K 16/32; A61K 31/517; A61P 35/00; A61K 47/6803; A61K 47/6857; A61K 45/06

(57) ABSTRACT

The present disclosure provides methods of treating cancer in a patient determined to have a HER2 mutation, such as an insertion mutation, by administering a third-generation tyrosine kinase inhibitor, such as poziotinib, in combination with a HER2 anti-body-drug conjugate.

This application claims the benefit of U.S. Provisional Application No. 62/784,084, filed Dec. 21, 2018, the entirety of which is incorporated herein by reference.

### BACKGROUND

### 1. Field

The present invention relates generally to the field of molecular biology and medicine. More particularly, it concerns methods of treating cancer patients with a combination therapy.

### 2. Description of Related Art

Human epidermal growth factor receptor 2 (HER2) amplifications occur in many cancer types and targeted agents such as trastuzumab, pertuzumab, trastuzumab emtansine (T-DM1), lapatinib and neratinib have been shown to improve clinical outcomes compared to chemotherapy alone. To date, several HER2 targeting agents are FDA approved for treating HER2 amplified disease in breast and gastric cancers. Activating mutations of HER2 have been reported in many cancer types; however, currently, there are no FDA approved targeted therapies for cancers harboring HER2 mutations.

Recent clinical studies of targeted agents for HER2 mutant cancers have focused on covalent second-generation tyrosine kinase inhibitors (TKIs) such as afatinib, neratinib, dacomitinib. The SUMMIT pan-cancer study reported that patients receiving neratinib had an objective response rate (ORR) of less than 15% for all HER2 mutations (Hyman et al., 2018). However, across multiple studies, when patients were stratified by cancer type, breast cancer patients have had ORRs of 12.5%-32% to neratinib; whereas lung cancer patients had 0%-4% response rates to neratinib as a single agent (Mazieres et al., 2015), demonstrating cancer specific differences in efficacy of HER2 inhibition. Interestingly, within a single cancer type, HER2 targeted agents appear to elicit variant-specific differences. In the SUMMIT trial, patients with HER2 kinase domain point mutations had an ORR of 21.4%, whereas patients with exon 20 insertions had an ORR of 7.1% to neratinib. Furthermore, dacomitinib had an ORR of 11.5% for HER2 mutant NSCLC, but no responses occurred among patients bearing the HER2 exon 20 insertion mutation, Y772dupYVMA (Kris et al., 2015); and in two separate studies of afatinib, NSCLC patients with exon 20 insertion mutations had response rates of 18.2% and 18.8% to afatinib.

Studies of HER2 monoclonal antibodies and drug-antibody conjugates revealed similar results. The pan-cancer study MyPathway tested the efficacy of the combination of anti-HER2 monoclonal antibodies trastuzumab and pertuzumab in 35 different tumor types and reported an ORR of 11% for all HER2 mutations and cancer types (Hainsworth et al., 2018). In this study, only 21% of NSCLC patients and one biliary cancer patient responded among the 35 tumor types included. In addition, in a pan-HER2 mutant NSCLC study testing the efficacy of T-DM1, patients harboring exon 20 insertion mutations had an ORR of 54.5%, but patients with exon 19 mutations did not have partial responses (Li et al., 2018). These cancer specific and variant specific differences in patient outcomes demonstrate the unmet need for a detailed and systematic understanding of the landscape of HER2 mutations across cancer types, and the identification of effective therapies for the various HER2 mutations identified.

Pre-clinical studies of HER2 activating mutations have also reported differential sensitivity to various TKIs. Studies of mutations within the HER2 extracellular domain have shown that these mutations are associated with resistance to non-covalent inhibitors such as lapatinib, yet robust sensitivity to covalent TKIs including neratinib, afatinib, and osimertinib (Greulich et al., 2012) while mutations within exon 19 demonstrate varying sensitivity to lapatinib and covalent inhibitors. Furthermore, studies have demonstrated that HER2 exon 20 mutations have extensive resistance to non-covalent and covalent TKIs such as osimertinib, nazartinib, rociletinib, and olmutinib (Bose et al., 2013).

Interestingly, covalent quinazolinamine-based TKIs neratinib, afatinib, and dacomitinib, induce differential responses to individual HER2 exon 20 mutations (Kosaka et al., 2017); however, only uncommon HER2 mutations demonstrated sensitivity to these TKIs at clinically relevant concentrations. More recently, it was reported that poziotinib effectively inhibited HER2 exon 20 insertion mutations at concentrations achievable in patients; and poziotinib treatment induced a radiological response in one patient harboring a HER2 exon 20 mutation (Robichaux et al., 2018). Nevertheless, a single HER2 TKI has not been identified to target the most common variants of HER2 mutant cancers.

### SUMMARY

Embodiments of the present disclosure provides methods and compositions for treating cancer in patients comprising a combination of a tyrosine kinase inhibitor (TKI) and a HER2 antibody. In one embodiment, the present disclosure provides a method of treating a cancer comprising administering an effective amount of a tyrosine kinase inhibitor (TKI) and a HER antibody-drug conjugate to the subject. In some aspects, the cancer is lung cancer. For example, the lung cancer is non-small cell lung cancer (NSCLC). In some aspects, the subject is human.

In some aspects, the HER2 antibody is trastuzumab. In particular aspects, the HER2 antibody-drug conjugate is trastuzumab emtansine (T-DM1).

In certain aspects, the TKI is a Quinazolinamine-based TKI, such as poziotinib, afatinib, neratinib, dacomitinib, or tarloxotinib. In particular aspects, the Quinazolinamine-based TKI is poziotinib. In specific aspects, the poziotinib is administered at a low dose, such as 1-16 mg, such as less than 16 mg, particularly less than 10 mg, such as 2, 3, 4, 5, 6, 7, 8, or 9 mg.

In particular aspects, the subject is administered poziotinib and T-DM1. In specific aspects, the subject is administered a single dose of T-DM1.

In some aspects, the cancer is a HER2 mutant cancer. For example, the HER2 mutant comprises activating mutations of HER2 within the tyrosine kinase domains spanning exons 19-21. In certain aspects, the HER2 mutant cancer comprises one or more mutations selected from the group consisting of L755S, L755P, D769H, D769Y, V773M, V777L, Y772dupYVMA, G776delinsVC, G776delinsVV, G776delinsLC, G778insLPS, P780insGSP, L786V, V842I, and L869R. In certain aspects, the HER2 mutant cancer has an exon 19, exon 20, and/or exon 21 mutation. In some aspects, the HER2 mutant cancer has an exon 20 mutation, such as one or more point mutations, insertions, and/or deletions of 1-18 nucleotides between amino acids E770-R786 of HER2. In particular aspects, the exon 20 mutation is at residue V773, A775, G776, V777, G778, 5779, and/or P780. In particular aspects, the exon 20 mutation is an exon 20 insertion mutation, such as Y772dupYVMA, G778dupGSP, and/or G776delinsVC. In some aspects, the exon 19 mutation is at residue L755 or D769, such as L755P. In particular aspects, the exon 20 mutation is a point mutation, such as at residue C805, such as C805S. In certain aspects, the exon 21 mutation is a point mutation. In some aspects, the point mutation is at residue V842 or L869, such as V842I or L869R.

In some aspects, the poziotinib is administered orally. In certain aspects, the poziotinib is administered at a dose of 5-25 mg, such as a dose of 8 mg, 12 mg, or 16 mg. In certain aspects, the poziotinib is further defined as poziotinib hydrochloride salt. The poziotinib hydrochloride salt may be formulated as a tablet.

In some aspects, the TKI is administered prior to or after the HER2 antibody-drug conjugate, such as 1 day, 2 days, 3 days, 4 days, 5 days, 6 days, 7 days, 2 weeks, 3 weeks, 1 month or more apart. the TKI is administered simultaneously with the HER2 antibody-drug conjugate.

In certain aspects, the poziotinib and/or the T-DM1 are administered intravenously, subcutaneously, intraosseously, orally, transdermally, in sustained release, in controlled release, in delayed release, as a suppository, or sublingually.

In additional aspects, the method further comprises administering an additional anti-cancer therapy. In some aspects, the additional anti-cancer therapy is chemotherapy, radiotherapy, gene therapy, surgery, hormonal therapy, anti-angiogenic therapy or immunotherapy.

A pharmaceutical composition comprising a TM and a HER antibody-drug conjugate. In particular aspects, the TKI is poziotinib and the HER antibody-drug conjugate is T-DM1.

A method of predicting a response to a TKI in combination with a HER2 antibody-drug conjugate in a subject having a cancer comprising detecting a HER2 mutation in a genomic sample obtained from said subject, wherein if the sample is positive for the presence of the HER2 mutation, then the patient is predicted to have a favorable response to the poziotinib in combination with the HER2 antibody-drug conjugate an anti-cancer therapy.

The HER2 mutation may comprises activating mutations of HER2 within the tyrosine kinase domains spanning exons 19-21. In certain aspects, the HER2 mutant cancer comprises one or more mutations selected from the group consisting of L755S, L755P, D769H, D769Y, V773M, V777L, Y772dupYVMA, G776delinsVC, G776delinsVV, G776delinsLC, G778insLPS, P780insGSP, L786V, V842I, and L869R. In certain aspects, the HER2 mutant cancer has an exon 19, exon 20, and/or exon 21 mutation. In some aspects, the HER2 mutant cancer has an exon 20 mutation, such as one or more point mutations, insertions, and/or deletions of 1-18 nucleotides between amino acids E770-R786 of HER2. In particular aspects, the exon 20 mutation is at residue V773, A775, G776, V777, G778, 5779, and/or P780. In particular aspects, the exon 20 mutation is an exon 20 insertion mutation, such as Y772dupYVMA, G778dupGSP, and/or G776delinsVC. In some aspects, the exon 19 mutation is at residue L755 or D769, such as L755P. In particular aspects, the exon 20 mutation is a point mutation, such as at residue C805, such as C805S. In certain aspects, the exon 21 mutation is a point mutation. In some aspects, the point mutation is at residue V842 or L869, such as V842I or L869R.

In certain aspects, the genomic sample is isolated from saliva, blood, urine, normal tissue, or tumor tissue. In some aspects, the presence of a HER2 mutation is determined by nucleic acid sequencing or PCR analyses. In some aspects, a favorable response to the TKI in combination with the HER antibody-drug conjugate comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, or increased patient survival.

In some aspects, the TKI is poziotinib and the HER antibody-drug conjugate is T-DM1. In some aspects, the method further comprising administering poziotinib in combination with T-DM1 to said subject predicted to have a favorable response.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: HER2 mutations occur in a variety of cancer types with mutational hotspots occurring across the receptor. Pie chart of frequency of HER2 mutation location reported in cBioportal and MD Anderson databases (N=2338).

FIGS. 2A-2D: HER2 mutation hotspots vary by cancer type. (A) Lollipop plot of the 11 most common HER2 mutations across all cancers reported in cBioPortal and MD Anderson (N=2338 HER2 mutations). Length of bars are relative to frequency of mutation. (B-D) Lollipop plots of the most common (>3%) HER2 mutations across NSCLC (B, N=177), Breast cancer (C, N=143), and Colorectal cancer (D, N=219) in cBioPortal and MD Anderson databases; length of bars are relative to frequency of mutation reported.

FIGS. 3A-3C: The most common HER2 variants in the tyrosine kinase domain are activating mutations. Cell viability of stable Ba/F3 cell lines expressing HER2 exon 19 (A), HER2 exon 20 (B), and HER2 exon 21 (C) mutations grown in IL-3 free conditions for 14 days. Cell viability was determined every 3 days by the Cell Titer Glo assay. The mean±SEM is plotted for each cell line (n=3 biologically independent experiments).

FIGS. 4A-4F: Poziotinib was the most potent inhibitor tested for HER2 mutations in Ba/F3 cells. (A) Heatmap of log IC₅₀ values calculated in GraphPad for Ba/F3 cells stably expressing the indicated mutations and drugs after 72 hours of drug treatment. Cell viability was determined by the Cell Titer Glo assay (N≥3). Average IC₅₀ values for all Ba/F3 cell lines expressing HER2 mutations (B), HER2 exon 19 mutant cell lines (C), HER2 exon 20 mutant cell lines (D), or HER2 exon 21 mutant cell lines (E) after drug treatment for 72 hours with neratinib, tarloxotinib-TKI, and poziotinib. Bars are representative of mean±SEM (N≥3). (C-E) One way ANOVA with Dunn's multiple comparisons test was used to determine statistical significance between groups. (F) Average IC₅₀ values of Ba/F3 cells expressing L755S or L755P with indicated inhibitors. Dots are representative of mean±SEM (N≥3). Statistical significance was determined by a paired t-test.

FIGS. 5A-5D: Molecular dynamics simulations of HER2 mutants reveal possible mechanisms for decreased drug sensitivity for Y772dupYVMA and L755P mutations. (A) α-C-helix positions for the HER2 V777L and Y772dupYVMA exon 20 mutants during the 150 ns accelerated molecular dynamics simulations. (B) Fractional population of molecular dynamics snapshots for the HER2 exon 20 mutants in the α-C-helix “in” vs. “out” conformations. (C) Molecular dynamics snapshots of the V777L (white backbone, light green P-loop) and Y772dupYVMA (grey backbone, dark green P-loop) mutants. There are minor differences in P-loop and kinase hinge conformations, but a significant shift in α-C-helix position (“out” position for V777L in blue, “in” position for Y772dupYVMA in purple). (D) Molecular dynamics snapshots of L755P (white backbone, light green P-loop, yellow hinge, blue α-C-helix) and L755S (grey backbone, dark green P-loop, orange hinge, purple α-C-helix) HER2 mutants. The L755P mutant lacks a backbone hydrogen bond with V790, leading to destabilization of the kinase hinge and contraction of the P-loop towards the binding site.

FIGS. 6A-6F: Human cell lines expressing HER2 mutations are also most sensitive to poziotinib. Dose response curves of MCF10A cells expressing exon 20 insertion mutations, HER2 G776delinsVC (A), HER2 Y772dupYVMA (B), HER2 G778dupGSP (C), treated with indicated inhibitors for 72 hours. (D) Bar graph of MCF10A HER2 selectivity index. IC₅₀ values of mutant cell lines were divided by average IC₅₀ value of HER2 WT expressing cell line for each indicated drug. Dots are representative of mean±SEM for each cell line and bars are representative of mean±min/max of all three cell lines (N≥3 for each cell line). (E) Dose response curve of CW-2 large intestine cells harboring HER2 exon 19 mutation L755S treated with indicated inhibitors for 72 hours. (A-C, E) Curves are representative of mean±SEM, N=3. (F) Bar graph of CW-2 tumor volume at day 21. Mice were treated with vehicle control (N=5), 30 mg/kg neratinib (N=5), 20 mg/kg afatinib (N=5), or 5 mg/kg poziotinib (N=5) 5 days/week and tumors were randomized at 350 mm³, indicated by the dotted line. Dots are representative of individual tumors, and bars are representative of mean±SEM. Statistical significance was determined by one-way ANOVA.

FIGS. 7A-7D: NSCLC patients with HER2 mutations have a 42% confirmed response rate to poziotinib. (A) Waterfall plot of first 12 HER2 exon 20 patient responses on clinical trial NCT03066206. Objective partial responses are shown for patient 7, 8, 10, 11, and 12 (5), an unconfirmed response is shown for patient 9 (1), stable disease is shown for patients 3-6 (5), and progressive disease is for patient 1 (1). (B) Kaplan-meier plot of progression free survival of the first 12 HER2 exon 20 patients demonstrates the mPFS was 5.6 months as of November 2018. (C) CT scan of a patient with a HER2 Y772dupYVMA mutation 1 day before poziotinib treatment and 8 weeks after therapy. (D) PET scans of patient with HER2 L755P mutant NSCLC 1 day before and 4 weeks after poziotinib treatment. Patient had been previously treated and progressed through, platinum-based chemotherapy in combination with trastuzumab, Nivolumab, and anti TDM1, but had a −12% reduction in target lesions with poziotinib treatment. Patient remains with stable disease for >7 months.

FIGS. 8A-8G: Poziotinib treatment induces accumulation of HER2 on the cell surface, and combination of poziotinib and T-DM1 treatment potentiates anti-tumor activity. (A) FACS analysis of HER2 receptor expression on MC10A cell lines expressing HER2 Y772dupYVMA, HER2 G778dupGSP, and HER2 G776delinsVC after 24 hours of 10 nM poziotinib treatment. Bars are representative of mean±SEM, and significant differences were determined by students' t-test between DMSO and poziotinib treated groups. (B) Bar graphs of IC₅₀ values of MCF10A cell lines expressing HER2 Y772dupYVMA, HER2 G778dupGSP, and HER2 G776delinsVC treated with poziotinib, T-DM1 or poziotinib and indicated dose of T-DM1. Bars are representative of mean±SEM (n=3 independent experiments), and significant differences were determined by One-way ANOVA and Dunn's multiple comparison post-hoc. (C) Tumor growth curves of HER2 Y772dupYVMA NSCLC PDX treated with the indicated inhibitors. Poziotinib treatment was administered five days per week, and T-DM1 was administered once at the beginning of treatment. (D) Kaplan-Meier curve of progression free survival (PFS), where PFS is defined as tumor doubling from best response. Mantel-Cox Log rank test was used to determine significant differences between groups. Mice were censored at time of euthanasia. (E) Dot plot of percent change in tumor volume of mice treated with indicated inhibitors at day 14. (F) Chart of number of tumor bearing mice in each group at day 14 and day 30. (G) Spider plots of tumor volume of HER2 Y772dupYVMA mice treated with indicated inhibitors.

FIG. 9: Exon 20 insertion mutation diversity differs by cancer type. Pie charts of HER2 exon 20 insertion mutation frequency in all cancer (A), Lung cancer (B), Breast cancer (C), and other cancers (D).

FIGS. 10A-10B: Common HER2 mutations are constitutively phosphorylated and p-HER2 expression does not correlate with drug sensitivity. (A) Relative p-HER2 expression was determined by taking the ratio of p-HER2 over total HER2 as determined by ELISA. Bars are representative of the mean±SEM, and n=3. ND=below the limit of detection. (B) Correlation of the relative HER2 was plotted against poziotinib IC50 values for Ba/F3 HER2 mutant cell lines. Pearson correlations and p-values were determined by GraphPad Prism (n=3).

FIGS. 11A-11B: Molecular modeling reveals HER2 mutants differ in binding pocket size. (A) HER2 kinase domain exon 19, 20 and 21 protein backbone colored in blue, pink, and orange, respectively. The ligand from the template X-ray structure (PDB 3PP0) is rendered in green sticks and labels are provided for mutated residues/insertion locations. (B) Binding pocket volume profiles for the HER2 mutants taken from the accelerated molecular dynamics simulations. Highest binding is for HER2 V777L and HER2 L869R.

FIG. 12: Poziotinib inhibits p-HER2 in HER2 mutant cell lines. Western blot of MCF10A cells expressing G776delinsVC after 2 hours treatment of the indicated drugs and doses.

FIG. 13: Poziotinib inhibits tumor growth in a xenograft of exon 19 mutant colorectal cancer. CW-2 cells harboring a HER2 L755S mutation were injected into the flanks of 6 week old female nu/nu nude mice. When tumors reached 350 mm³ mice were randomized into 4 groups: 20 mg/kg afatinib, 5 mg/kg poziotinib, 30 mg/kg neratinib, or vehicle control. Tumor volumes were measured three times per week, and mice received drug Monday-Friday (5 days per week). Symbols are representative of the mean±SEM for each time point. Two-Way ANOVA with Tukey's multiple comparisons test was used to determine statistical significance. Asterisk indicate significance between vehicle and poziotinib (red) or neratinib (grey). P-values for each comparison are listed below beginning at 10 day when significant differences were first detected.

### DESCRIPTION OF ILLUSTRATIVE EMBODIMENTS

In the present studies, the frequency of the most common genomic variants of HER2 mutations across various malignancies was determined. Systematically, the activating potential of the 16 most frequent HER2 mutations was demonstrated and their drug sensitivity was evaluated across 11 commonly utilized EGFR and HER2 TKIs. It was found that exon 20 insertion mutations and the L755P (but not the L755S) mutation in exon 19 were refractory to many of the TKIs tested. Molecular dynamic modeling of drug resistant HER2 variants, L755P and exon 20 insertions, demonstrated that these mutations affect the conformational states of the receptor, reducing the overall size of the drug binding pocket. Furthermore, poziotinib was identified as a potent inhibitor of all HER2 mutations evaluated; and, poziotinib had clinical activity in NSCLC patients harboring the most resistant HER2 variants, exon 20 insertions and L755P. Lastly, it was shown that poziotinib-mediated cell surface receptor accumulation enhances T-DM1 activity that can be exploited to increase anti-tumor activity in vivo, leading to complete tumor regression in a PDX model of HER2 mutant NSCLC.

Thus, in certain embodiments, the present disclosure provides a combination therapy comprising a TKI and a HER2 antibody conjugate, such as poziotinib and T-DM1, for the treatment of cancer, such as HER2 mutant cancers including NSCLC.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, the stated value plus or minus 5%.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease. For example, a treatment may include administration of an effective amount of poziotinib.

“Prophylactically treating” includes: (1) reducing or mitigating the risk of developing the isease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “insertion(s)” or “insertion mutation(s)” refers to the addition of one or more nucleotide base pairs into a DNA sequence. In one example, HER2 exon 20 insertion mutation comprises one or more insertions of 3-18 nucleotides between amino acids 770-785.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

### II. HER2 MUTATIONS

Certain embodiments of the present disclosure concern determining if a subject has one or more HER2 mutation, such as HER2 exon 19, exon 20, or exon 21 mutations, such as an insertion, deletion, or point mutation. The subject may have 2, 3, 4, or more HER2 mutations. Mutation detection methods are known the art including PCR analyses and nucleic acid sequencing as well as FISH and CGH. In particular aspects, the HER2 mutations are detected by DNA sequencing, such as from a tumor or circulating free DNA from plasma.

Exemplary HER2 exon 19 mutations include mutations at L755 or D769, such as L755P. Exemplary HER2 exon 21 mutations include mutations at V842 or L869, such as V842I or L869R.

The HER2 exon 20 mutation may comprise one or more point mutations, insertions, and/or deletions of 1-18, such as 3-18, nucleotides between amino acids 770-786. The one or more HER2 exon 20 mutations may be at residue Y772, V773, A775, G776, V777, G778, 5779, and/or P780. The one or more HER2 exon 20 mutations may be A775insV G776C, A775insYVMA, G776V, G776C V777insV, G776C V777insC, G776del insVV, G776del insVC, P780insGSP, V777L, G778insLPS, and/or V773M. Particularly, the HER exon 20 insertion mutations may be Y772dupYVMA, G778dupGSP, and/or G776delinsVC.

The patient sample can be any bodily tissue or fluid that includes nucleic acids from the lung cancer in the subject. In certain embodiments, the sample will be a blood sample comprising circulating tumor cells or cell free DNA. In other embodiments, the sample can be a tissue, such as a lung tissue. The lung tissue can be from a tumor tissue and may be fresh frozen or formalin-fixed, paraffin-embedded (FFPE). In certain embodiments, a lung tumor FFPE sample is obtained.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine, tumor, or expectorant. The sample itself will typically include nucleated cells (e.g., blood or buccal cells) or tissue removed from the subject including normal or tumor tissue. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of HER2 mutations as described herein can be determined using methods known in the art. For example, gel electrophoresis, capillary electrophoresis, size exclusion chromatography, sequencing, and/or arrays can be used to detect the presence or absence of insertion mutations Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an insertion mutation as described herein. An insertion mutation can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

A set of probes typically refers to a set of primers, usually primer pairs, and/or detectably-labeled probes that are used to detect the target genetic variations (e.g., HER2 mutations) used in the actionable treatment recommendations of the present disclosure. The primer pairs are used in an amplification reaction to define an amplicon that spans a region for a target genetic variation for each of the aforementioned genes. The set of amplicons are detected by a set of matched probes. In an exemplary embodiment, the present methods may use TaqMan™ (Roche Molecular Systems, Pleasanton, Calif.) assays that are used to detect a set of target genetic variations, such as HER2 mutations. In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed.

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears et al., 1992), solid-phase sequencing (Zimmerman et al., 1992), sequencing with mass spectrometry such as matrix-assisted laser desorption/ionization time-of-flight mass spectrometry (MALDI-TOF/MS; Fu et al., 1998), and sequencing by hybridization (Chee et al., 1996; Drmanac et al., 1993; Drmanac et al., 1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC, Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

In one example, a method of identifying a HER2 mutation in a sample comprises contacting a nucleic acid from said sample with a nucleic acid probe that is capable of specifically hybridizing to nucleic acid encoding a mutated HER2 protein, or fragment thereof incorporating a mutation, and detecting said hybridization. In a particular embodiment, said probe is detectably labeled such as with a radioisotope (³H, ³²P, or ³³P), a fluorescent agent (rhodamine, or fluorescein) or a chromogenic agent. In a particular embodiment, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, said probes of the present disclosure are provided in a kit for identifying HER2 mutations in a sample, said kit comprising an oligonucleotide that specifically hybridizes to or adjacent to a site of mutation in the HER2 gene. The kit may further comprise instructions for treating patients having tumors that contain HER2 insertion mutations with poziotinib based on the result of a hybridization test using the kit.

In another aspect, a method for detecting an exon 20 mutation in a sample comprises amplifying from said sample nucleic acids corresponding to exon 20 of said HER gene, or a fragment thereof suspected of containing a mutation, and comparing the electrophoretic mobility of the amplified nucleic acid to the electrophoretic mobility of corresponding wild-type HER2 gene or fragment thereof. A difference in the mobility indicates the presence of a mutation in the amplified nucleic acid sequence. Electrophoretic mobility may be determined on polyacrylamide gel.

Alternatively, nucleic acids may be analyzed for detection of mutations using Enzymatic Mutation Detection (EMD) (Del Tito et al., 1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel electrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

### III. METHODS OF TREATMENT

Further provided herein are methods for treating or delaying progression of cancer in an individual comprising administering to the individual an effective amount of poziotinib, or a structurally similar inhibitor, and T-DM1 to a subject with cancer, such as a HER2 mutant cancer. The subject may be determined to have HER2 mutations, such as exon 19, 20, or 21 mutations, such as an exon 20 insertion, or a L755P mutation. The subject may have more than one HER mutation.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is non-small cell lung cancer.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

Certain embodiments concern the administration of poziotinib (also known as HM781-36B, HM781-36, and 1-[4-[4-(3,4-dichloro-2-fluoroanilino)-7-methoxyquinazolin-6-yl]oxypiperidin-1-yl]prop-2-en-1-one). Poziotinib is a quinazoline-based pan-HER inhibitor that irreversibly blocks signaling through the HER family of tyrosine-kinase receptors including HER1, HER2, and HER4. Poziotinib or structurally similar compounds (e.g., U.S. Pat. No. 8,188,102 and U.S. Patent Publication No. 20130071452; incorporated herein by reference) may be used in the present methods.

The poziotinib, such as poziotinib hydrochloride salt, may be administered orally, such as in a tablet. The poziotinib may be administered in a dose of 4-25 mg, such as at a dose of 5, 6, 7, 8, 9, 10, 11, 12, 13, 14, 15, 16, 17, 18, 19, 20, 21, 22, 23, or 24 mg. The dosing may be daily, every other day, every 3 days or weekly. The dosing may be on a continuous schedule, such as on 28 days cycles.

Trastuzumab emtansine also known as ado-trastuzumab emtansine and sold under the trade name Kadcyla, is an antibody-drug conjugate consisting of the humanized monoclonal antibody trastuzumab (Herceptin) covalently linked to the cytotoxic agent emtansine (DM1). Trastuzumab alone stops growth of cancer cells by binding to the HER2 receptor, whereas trastuzumab emtansine undergoes receptor-mediated internalization into cells, is catabolized in lysosomes where DM1-containing catabolites are released and subsequently bind tubulin to cause mitotic arrest and cell death. Trastuzumab binding to HER2 prevents homodimerization or heterodimerization (HER2/HER3) of the receptor, ultimately inhibiting the activation of MAPK and PI3K/AKT cellular signalling pathways. Because the monoclonal antibody targets HER2, and HER2 is only over-expressed in cancer cells, the conjugate delivers the cytotoxic agent DM1 specifically to tumor cells. The conjugate is abbreviated T-DM1. T-DM1 may be administered at a dose of 2-3 mg/kg, such as 3.6 mg/kg. The T-DM1 may be administered by intravenous infusion.

A. Pharmaceutical Compositions

Also provided herein are pharmaceutical compositions and formulations comprising poziotinib and T-DM1 and a pharmaceutically acceptable carrier, such as for subjects determined to have a HER2 exon 20 mutation, such as an exon 20 insertion.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve poziotinib and T-DM1 in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The poziotinib and T-DM1 may be administered before, during, after, or in various combinations relative to an additional cancer therapy, such as immune checkpoint therapy. The administrations may be in intervals ranging from concurrently to minutes to days to weeks. In embodiments where the poziotinib and T-DM1 is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below poziotinib and T-DM1 is “A” and an anti-cancer therapy is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129; International Patent Publication Nos. WO 01/14424, WO 98/42752, and WO 00/37504 (CP675,206, also known as tremelimumab; formerly ticilimumab); U.S. Pat. No. 6,207,156; Hurwitz et al., 1998; Camacho et al., 2004; and Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001014424, and WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. KIT

Also within the scope of the present disclosure are kits for detecting EGFR and/or HER2 exon 20 mutations, such as those disclosed herein. An example of such a kit may include a set of exon 20 mutation-specific primer. The kit may further comprise instructions for use of the primers to detect the presence or absence of the specific EFGR and/or HER2 exon 20 mutations described herein. The kit may further comprise instructions for diagnostic purposes, indicating that a positive identification of EGFR and/or HER2 exon 20 mutations described herein in a sample from a cancer patient indicates sensitivity to the tyrosine kinase inhibitor poziotinib or a structurally similar inhibitor and T-DM1. The kit may further comprise instructions that indicate that a positive identification of EGFR and/or exon 20 mutations described herein in a sample from a cancer patient indicates that a patient should be treated with poziotinib or a structurally similar inhibitor and T-DM1.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Treating HER2 Cancers

HER2 mutations occur most frequently in cancers of the bladder, stomach, and bile duct: To understand the diversity of HER2 mutations across cancer types, several databases were queried within cBioPortal (N=44,037) and MD Anderson Cancer Center (N=19,926).

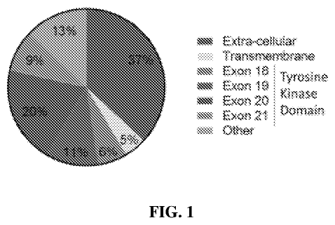

HER2 mutations occur most frequently in the tyrosine kinase domain of HER2: Next, the frequency of mutations was analyzed within the various regions of the HER2 receptor reported in cBioPortal and at MD Anderson. Across all cancer types, HER2 mutations occurred most frequently in the tyrosine kinase domain (46%) which included mutations in exon 20 (20%), exon 19 (11%), and exon 21 (9%) (FIG. 1); in addition, extra-cellular domain mutations made up 37% of HER2 mutations.

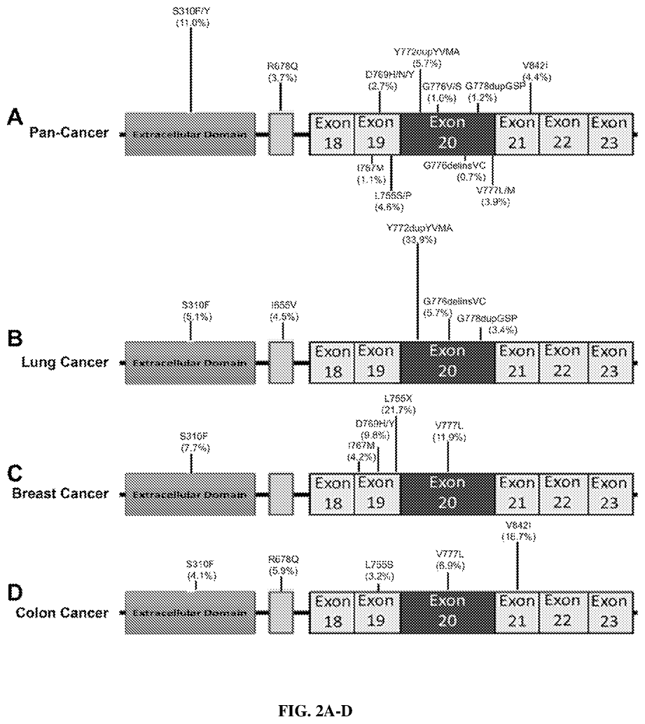

HER2 mutational hotspots vary by malignancy type: Across all cancers queried, the most common HER2 mutations were: S310F/Y (11.0%), Y772_A775dupYVMA (5.7%), L755P/S (4.6%), V842I (4.4%), and V777L/M (4.0%) (FIG. 2A). In lung cancer, the majority of HER2 mutations occurred within exon 20 (47%), with Y772_A775dupYVMA, comprising 34% of all HER2 mutations (FIG. 2B). In breast cancer, the majority of HER2 mutations occurred within exon 19 (37%), with L755 mutations being the most prevalent at 22% of HER2 mutations. However, unlike lung cancer where one variant was dominant, in breast cancer, there was more mutational diversity among exon 19 mutations (FIG. 2C). In colorectal cancer, HER2 mutations occurred most frequently in exon 21 (22%), with the V842I variant being the most prevalent (19%) (FIG. 2D).

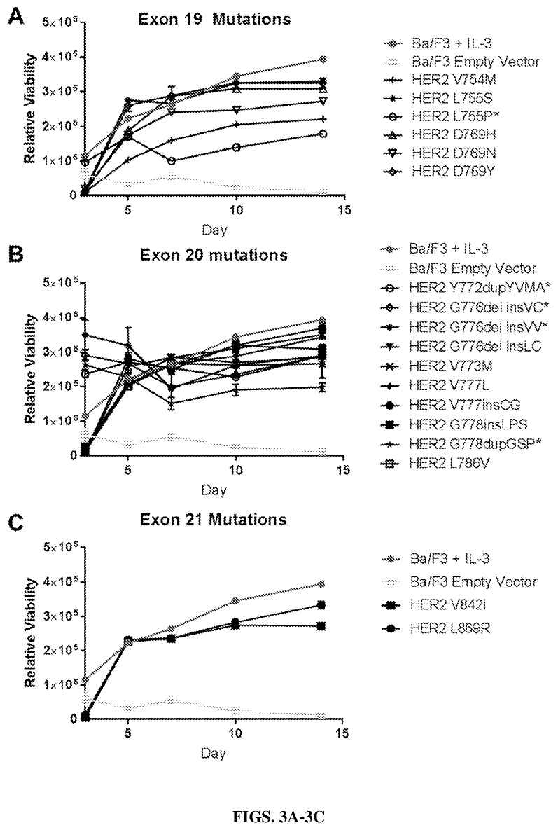

Frequently detected HER2 alterations are activating mutations: To assess the functional impact of common HER2 mutations, Ba/F3 cells were stably expressed with the 16 most frequently detected HER2 mutation across exons 19, 20 and 21. All 16 HER2 mutations tested were found to induce IL-3 independent survival of Ba/F3 cells (FIGS. 3A-C) and expressed phosphorylated HER2, suggesting that these mutations result in receptor activation.

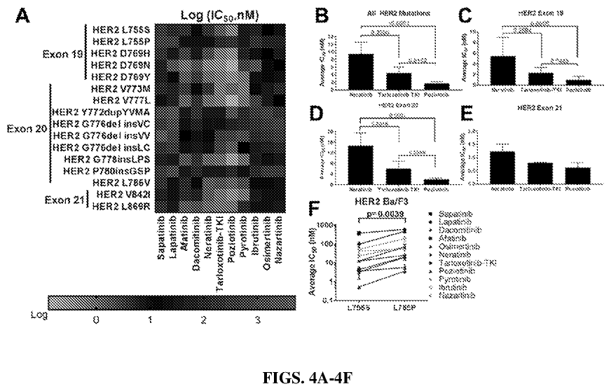

Poziotinib was the most TKI tested and inhibited the most common HER2 mutations in vitro: While recent reports highlight the effectiveness of covalent quinazolinamine-based TKIs (i.e. afatinib, dacomitinib, poziotinib, neratinib) in pre-clinical models of HER2 mutant disease (Nagano et al., 2018), clinical studies of afatinib, dacomitinib, and neratinib have had low ORRs, cancer specific and variant specific differences in patient outcomes. To systematically evaluate drug sensitivity across the most commonly detected HER2 variants, a panel of HER2 mutant Ba/F3 cells were screened against 11 covalent and non-covalent EGFR and HER2 TKIs. HER2 mutants showed robust resistance to non-covalent inhibitors, lapatinib and sapatinib (FIG. 4A). Covalent TKIs osimertinib, ibrutinib, and nazartinib were not effective in inhibiting cell viability in cells expressing exon 20 mutations; however, these TKIs did demonstrate activity against cells expressing D769 exon 19 variants and exon 21 variants (FIG. 4A). By comparison, covalent, quinazolinamine-based TKIs, afatinib, neratinib, dacomitinib, tarloxotinib-TKI, and poziotinib, had inhibitory activity for HER2 mutants across all three exons (FIG. 4A). Across all HER2 mutation variants and TKIs tested, poziotinib had the lowest average IC₅₀ and was significantly more effective in reducing cell viability than neratinib and tarloxotinib-TKI (p<0.001 and p=0.018, respectively, FIG. 4B). In addition, while poziotinib was more efficacious than either neratinib or tarloxotinib-TKI against HER2 exon 19 and 20 mutations, there was no significant difference in average IC₅₀ for exon 21 mutants (FIGS. 4C-E), suggesting that mutation location impacts drug binding. Furthermore, within exon 19, L755S and L755P variants had significant differences in drug sensitivity across all TKIs tested (FIG. 4F), indicating that specific amino acid changes at this site influenced drug binding affinity.

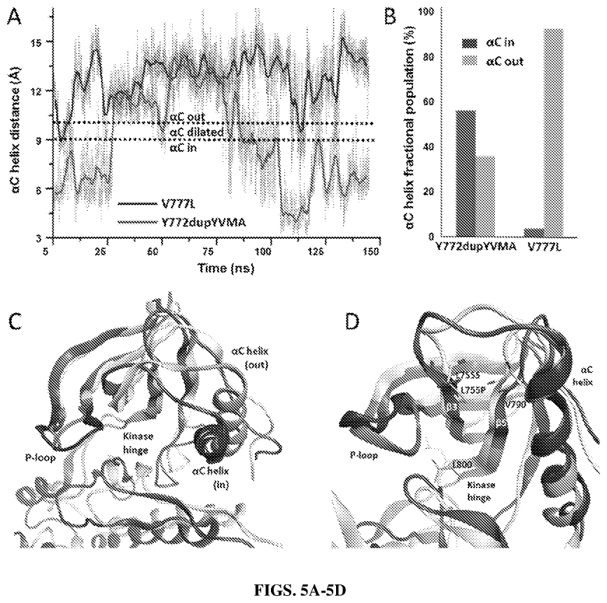

HER2 mutation location and amino acid change effects drug binding affinity: To further understand how the location of the mutation and the amino acid change can affect drug binding affinity and inhibitory efficacy, molecular dynamics simulations were used to investigate how these mutations impact the structure and dynamics of the HER2 kinase domain. Molecular models of the L755S, L755P, Y772dupYVMA, V777L, and L869R HER2 mutants were constructed using a publicly available X-ray structure (PDB 3PP0) as a template and subjected to accelerated molecular dynamics to increase protein conformational sampling. The range of protein conformations sampled, particularly in regards to the P-loop and α-C-helix positions, varied among these HER2 mutants. Differences were clearly evident even between exon 20 mutations, especially in the α-C-helix region, where the duration of the conformation of the α-C-helix varied between the “in” (the active conformation with a smaller binding pocket), and the “out” (the inactive conformation with a larger binding pocket). The V777L mutant heavily sampled the “out” conformation while the Y772dupYVMA mutant sampled both the “in” and “out” conformations (FIG. 5A). Overall, these differences in conformational state resulted in the Y772dupYVMA mutant residing in the “in” conformation 10-times more often than the V777L mutant (FIG. 5B), and, on average, a smaller binding pocket size for Y772dupYVMA compared to V777L (FIG. 5C). In addition, the smaller binding pocket of the Y772dupYVMA may be the cause of the weaker potency of neratinib against the Y772dupYVMA compared to the V777L since neratinib contains a pyridyl ring oriented towards the α-C-helix.

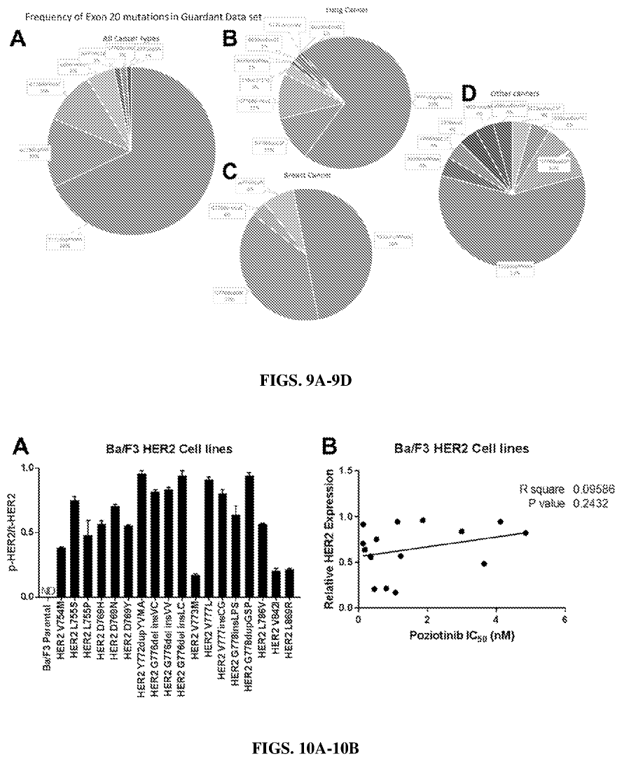

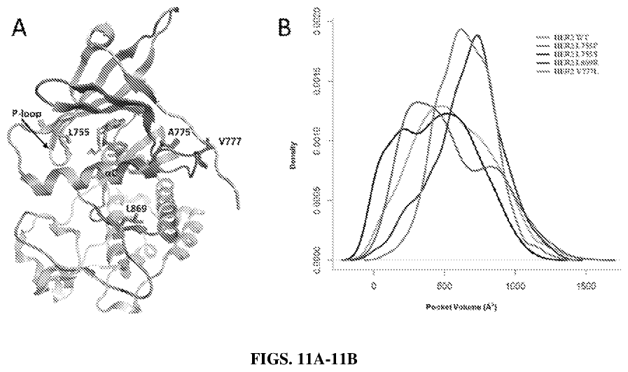

Further analysis of the HER2 mutant binding pocket volumes (FIG. 10B) demonstrated that mutations at the same residue can have drastically different effects on protein conformation. In particular, the proline residue of the L755P mutation lacks a hydrogen bond donor which breaks a backbone hydrogen bond between the β3 and β5 strands between L755 and V790, respectively. The lack of stabilization between these two β-strands resulted in destabilization of the β-sheet and a structural rearrangement in the kinase hinge region (FIG. 5D). In particular the L800 residue of L755P protruded into the active site and reduced the pocket size considerably. Changes in the β3 strand conformation also caused the P-loop to collapse inward, further reducing pocket volume and making this mutant less sensitive to most TKIs. Furthermore, the changes in hinge mobility may also play a role in kinase activation. These distinct changes in the L755P mutant confirmation contrasted with the behavior of the L755S mutant, which had a conformational and pocket volume profile that is more similar to wild-type HER2.

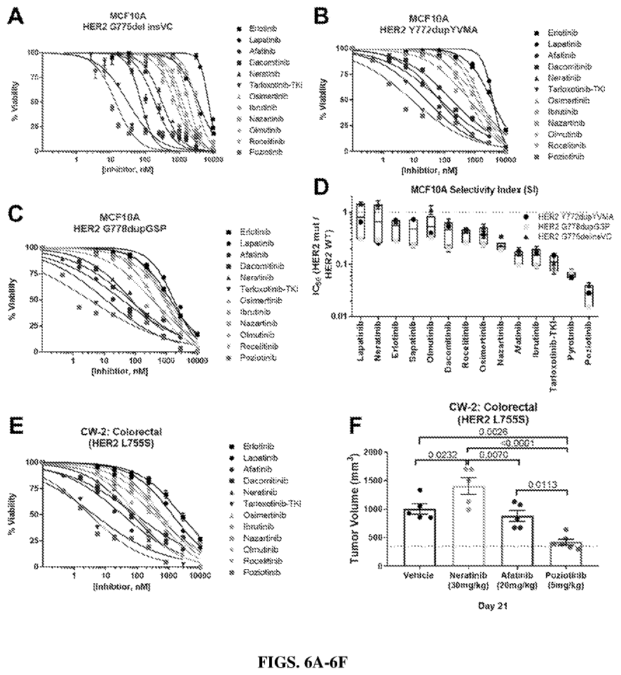

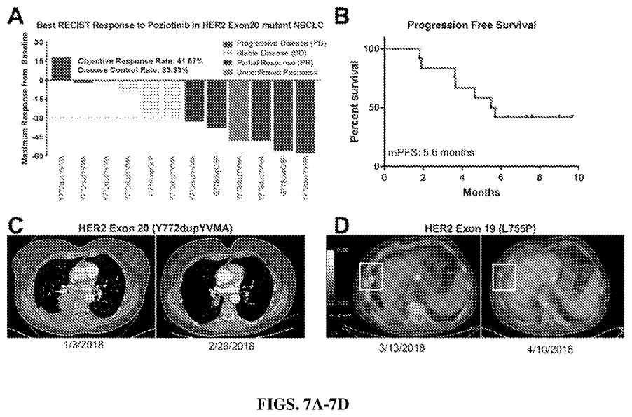

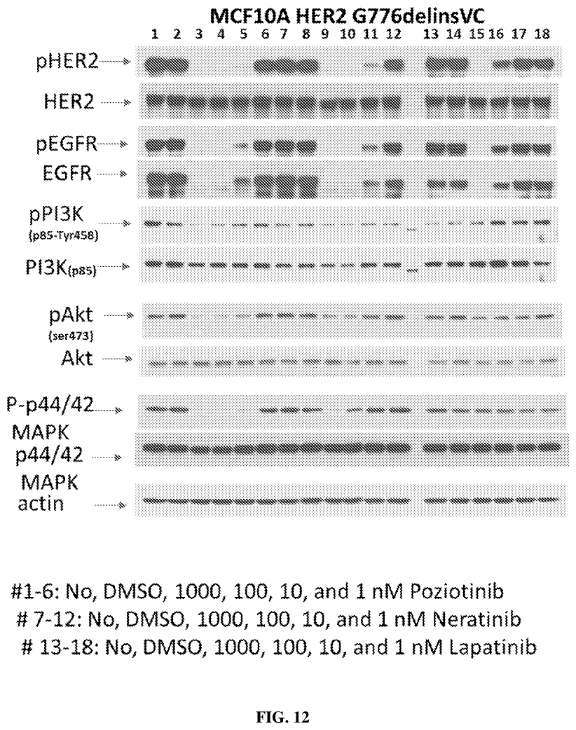

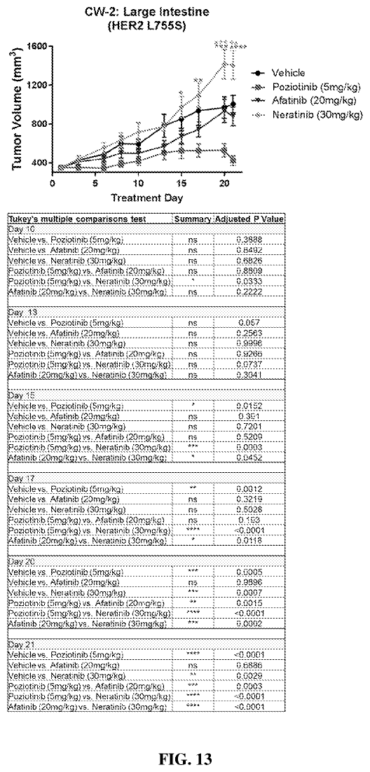

HER2 mutant human cancer cell lines showed enhanced sensitivity to poziotinib: Clinical studies testing HER2 inhibitors have revealed cancer type specific differences in drug sensitivity (Hyman et al., 2018). To determine whether covalent, quinazolinamine-based TKIs have marked activity in models of HER2 mutant disease, a panel of EGFR/HER2 TKIs were tested in human cancer cell lines. Pre-neoplastic MCF10A mammary epithelial cells were transfected with HER2 exon 20 mutations and evaluated in vitro sensitivity to 12 EGFR/HER2 TKIs. MCF10A cells expressing G776del insVC, Y772dupYVMA, or G778dupGSP HER2 mutations were most sensitive to poziotinib, with IC₅₀ values of 12 nM, 8.3 nM, 4.5 nM, respectively (FIGS. 6A-C). In comparison, tarloxotinib-TKI and neratinib yielded average IC₅₀ values of 21 nM and 150 nM, respectively (FIGS. 6A-C), indicating that poziotinib is 2.6 and 19 times more potent than tarloxotinib-TKI and neratinib, respectively (p<0.001). Furthermore, Western blotting of MCF10A HER2 G776delinsVC cells with poziotinib and neratinib showed that poziotinib, but not neratinib, completely inhibits p-HER2 at 10 nM (FIG. 12A). Since wild-type (WT) HER2 does not transform Ba/F3 cells to grow independent of IL-3, MCF10A cells were used to determine the selectivity of the TKIs for mutant HER2 compared to WT HER2. To this end, the selectivity index (SI, IC₅₀ value mutant/IC₅₀ value WT) was calculated for each inhibitor, and it was found that poziotinib was the most mutant selective TKI tested in MCF10A cell lines (SI=0.028), followed by pyrotinib (SI=0.063) and tarloxotinib-TKI (SI=0.111), (FIG. 6D). Consistent with the data obtained using Ba/F3 cells (FIG. 3C), in a model of HER2 exon 19 mutant colorectal cancer (CW-2), differences in sensitivity between poziotinib, tarloxotinib-TKI, and neratinib were less dramatic, albeit significant (p=0.02 and p=0.0004), with average IC₅₀ values of 3.19 nM, 4.24 nM, and 68.8 nM, respectively (FIG. 6E). Furthermore, in a xenograft mouse model of CW-2 colorectal cells, at day 21, poziotinib (5 mg/kg) treated animals had showed a reduction of 58% in tumor volume compared to the vehicle treated group (p=0.011). In comparison, neratinib (30 mg/kg) treated animals showed an increased tumor volume (28%) compared to vehicle control (p=0.023), and afatinib (20 mg/kg) treatment did not significantly affect tumor growth compared to vehicle control (FIG. 6F, FIG. 13).

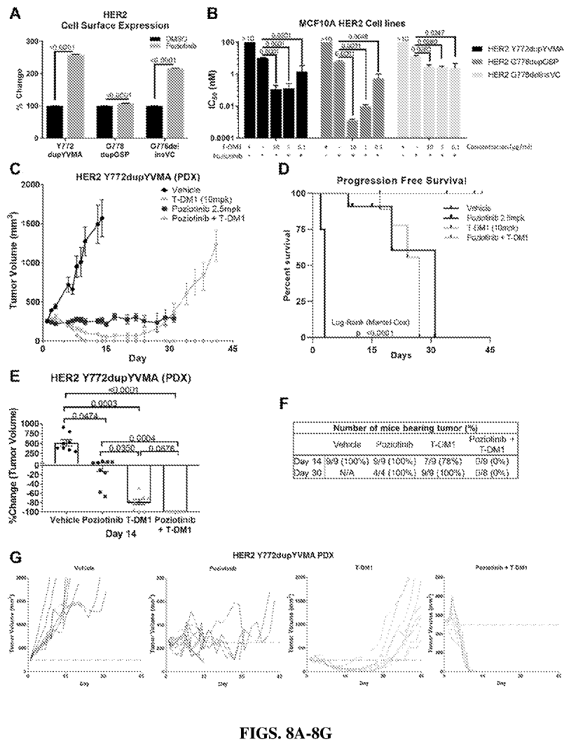

Poziotinib has anti-tumor activity in NSCLC patients with HER2 mutations: Based on these preclinical data and previously published work on exon 20 mutations (Robichaux et al., 2018), an investigator-initiated, phase II clinical trial was begun of poziotinib in EGFR and HER2 exon 20 mutant NSCLC (NCT03066206). Patients were treated with poziotinib 16 mg orally daily until progression, death, or withdrawal. Objective response was evaluated every eight weeks, based on RECIST v1.1. Of the first 12 evaluable patients harboring HER2 exon 20 insertion mutations, 6/12 (50%) patients had a best response of partial response (PR); this response was confirmed by a repeat scan 2 months later in 5/12 (confirmed objective response rate, 42%) (FIG. 7A). Of these patients, two patients had progressive disease (PD) at first response evaluation, resulting in a disease control rate (DCR) of 83%. As of November 2018, 7 of the twelve patients had progressed, and the median PFS for the first twelve patients was 5.7 months (FIG. 7B). All patients included in the study thus far harbored one of the two most common exon 20 insertions, Y772dupYVMA and G778dupGSP (FIG. 7A). Representative images of one NSCLC patient with an Y772dupYVMA mutation pre- and post-treatment (8 weeks) showed robust tumor shrinkage in the right lung (FIG. 7C). Patient characteristics including number of previous lines of treatment can be found in Table 3. In addition, one heavily pre-treated NSCLC patient harboring a HER2 exon 19 point mutation, L755P, was treated on a compassionate care use protocol (C-IND18-0014). The patient was treated with 16 mg poziotinib daily and had tumor shrinkage at four weeks (FIG. 7D, white box). The patient had stable disease (SD) per RECIST v1.1 (−12% reduction in target legions). The patient remained on poziotinib with disease control for more than 7 months until imaging revealed disease progression and poziotinib was discontinued. The patient was clinically well at the end of poziotinib treatment and proceeded to receive further systemic therapy.

Combination of poziotinib and T-DM1 treatment potentiates anti-tumor activity: Previous studies of HER2 TKI lapatinib in HER-positive breast cancer models and EGFR inhibitors in EGFR mutant NSCLC models have shown that TKI treatment results in an increase of receptor accumulation on the cell surface, and that increased cell surface HER2/EGFR increases sensitivity to antibody-dependent cellular cytotoxicity (ADCC). To determine if poziotinib treatment increases total HER2 receptor expression on the cell surface cell surface HER2 expression was analyzed by FACS after 24 hours of poziotinib treatment. It was found that, on average, poziotinib treatment increased cell surface HER2 expression 2-fold (FIG. 8A, p<0.0001). Next, it was tested if combination of poziotinib and T-DM1 would decrease cell viability in vitro, and it was found that while T-DM1 alone did not inhibit cell viability of MCF10A HER2 mutant cell lines, combination of T-DM1 with poziotinib resulted in lower IC₅₀ values than either agent alone (FIG. 8B). To validate these findings in vivo, the combination of low dose poziotinib with a single dose of T-DM1 was tested in a HER2 mutant NSCLC PDX model (HER2 Y772dupYVMA). It was found that at day 14, the combination of low dose poziotinib (2.5 mg/kg) and a single dose of T-DM1 (10 mg/kg) resulted in complete tumor regression in 8/8 mice, compared to 2/9 mice receiving T-DM1 alone or 0/9 mice receiving low dose poziotinib (FIGS. 8C-D, p<0.0001). By four weeks, tumor growth resumed in mice receiving T-DM1 alone; however, in all mice receiving combination treatment there was no evidence of tumor reoccurrence (FIG. 8E).

The present studies showed that HER2 mutations occur in various tumor types although the specific mutational hotspots vary by malignancy. Moreover, sensitivity to HER2 TKIs is heterogeneous across mutation location, with HER2 exon 20 insertions and L755P mutations being resistant to the majority of HER2 TKIs, likely due to the reduced volume of the drug binding pocket. Furthermore, poziotinib was identified as a potent, pan-HER2 mutant-selective inhibitor with clinical efficacy in NSCLC patients bearing HER2 exon 20 insertions and L755P mutations. Lastly, it was established that poziotinib treatment induced accumulation of HER2 on the cell surface, and that combination of poziotinib and T-DM1 treatment enhanced anti-tumor activity in vitro and in vivo.

TABLE 1
Vectors used to generate stable cell lines.

| Name | Mutation | Vendor |
|---|---|---|
| HER2 L755S | c.2264T > C | Created by Bioinnovatise from pBabe-puro |
| | | HER2 WT from Addgene (#40978) |
| HER2 D769H | c.2305G > A | Created by Bioinnovatise from pBabe-puro |
| | | HER2 WT from Addgene (#40978) |
| HER2 D769N | c.2305G > C | Created by Bioinnovatise from pBabe-puro |
| | | HER2 WT from Addgene (#40978) |
| HER2 D769Y | c.2305G > T | Created by Bioinnovatise from pBabe-puro |
| | | HER2 WT from Addgene (#40978) |
| HER2 | c.2323_2324insTATGTCATGGCT | Purchased from Addgene (#40982) |
| Y772dupYVMA | | |
| HER2 | c.2326_2328insTCT | Created by Bioinnovatise from pBabe-puro |
| G776del insVC | | HER2 WT from Addgene (#40978) |
| HER2 | c.2327delinsTTGT | Created by Bioinnovatise from pBabe-puro |
| G776del insVV | | HER2 WT from Addgene (#40978) |
| HER2 | c.2326G > TTGT | Created by Bioinnovatise from pBabe-puro |
| G776del insLC | | HER2 WT from Addgene (#40978) |
| HER2 V773M | c.2317G > A | Created by Bioinnovatise from pBabe-puro |
| | | HER2 WT from Addgene (#40978) |
| HER2 V777L | c.2329G > T | Created by Bioinnovatise from pBabe-puro |
| | | HER2 WT from Addgene (#40978) |
| HER2 | c.2332_2333insGGCTCCCCA | Created by Bioinnovatise from pBabe-puro |
| G778insLPS | | HER2 WT from Addgene (#40978) |
| HER2 | c.2339_2340insTGGCTCCCC | Created by Bioinnovatise from pBabe-puro |
| P780insGSP | | HER2 WT from Addgene (#40978) |
| HER2 L786V | c.2356C > G | Created by Bioinnovatise from pBabe-puro |
| | | HER2 WT from Addgene (#40978) |
| HER2 V842I | c.2524G > A | Created by Bioinnovatise from pBabe-puro |
| | | HER2 WT from Addgene (#40978) |
| HER2 L869R | c.2606T > G | Created by Bioinnovatise from pBabe-puro |
| | | HER2 WT from Addgene (#40978) |

TABLE 2
Patient Characteristics and number of prior lines of therapy.

| | | # of prior | |
|---|---|---|---|
| Age | Sex | lines | Mutation |
| 57 | F | 1 | Y772_A775dupYVMA |
| 64 | F | 6 | Y772_A775dupYVMA |
| 54 | F | 1 | A775_G776insYVMA |
| 59 | F | 0 | Y772_A775dupYVMA |
| 58 | F | 3 | Y772_A775dupYVMA |
| 60 | F | 1 | G778_P780dupGSP |
| 61 | F | 3 | G778_P780dupGSP |
| 62 | F | 0 | A775_G776insYVMA |
| 55 | F | 2 | G778_P780dupGSP |
| 61 | M | 4 | Y772_A775dupYVMA |
| 63 | M | 1 | Y772_A775dupYVMA |
| 60 | F | 3 | Y772_A775dupYVMA |

### Example 2—Materials and Methods

Analysis of HER2 mutation prevalence and variant frequency: To determine the frequencies of each HER2 mutation reported in databases from MD Anderson Cancer Center and cBioPortal, each database was queried individually, then frequencies were weighted by the total number of patients in each database and are reported as weighted averages. To determine the frequency of HER2 mutations across cancer types in cBioPortal, all non-overlapping studies were selected and exported. For overlapping studies, only the largest dataset was used. To determine HER2 mutation frequencies at MD Anderson Cancer, the Institute for Personalized Cancer Therapy database was queried for all HER2 mutations independent of cancer type.

Ba/F3 Cell line generation and IL-3 deprivation: Ba/F3 cell lines were established as previously described (Robichaux et al., 2018). Briefly, stable Ba/F3 cell lines were generated by retroviral transduction of Ba/F3 cell line for 12 hours. Retroviruses were generated by transfecting pBabe-Puro based vectors summarized in Table 1 (Addgene and Bioinnovatise) into Phoenix 293T-ampho cells (Orbigen) using Lipofectamine 2000 (Invitrogen). Three days after transduction, 2 μg/ml puromycin (Invitrogen) was added to the RPMI media. After 5 days of selection, cells were stained with FITC-HER2 (Biolegend) sorted by FACS. Cell lines were then grown in the absence of IL-3 for two weeks and cell viability was assessed every three days using the Cell Titer Glo assay (Progema). Resulting stable cell lines were maintained in RPMI-1640 media containing 10% FBS without IL-3.

Cell Viability Assay and ICso Estimation: Cell viability was determined using the Cell Titer Glo assay (Promega) as previously described (Robichaux et al., 2018). Briefly, 2000-3000 cells per well were plated in 384-well plates (Greiner Bio-One) in technical triplicate. Cells were treated with seven different concentrations of tyrosine kinase inhibitors or vehicle alone at a final volume of 40 μL per well. After 3 days hours, 11 μL of Cell Titer Glo was added to each well. Plates were shaken for 15 minutes, and bioluminescence was determined using a FLUOstar OPTIMA multi-mode micro-plate reader (BMG LABTECH). Bioluminescence values were normalized to DMSO treated cells, and normalized values were plotted in GraphPad Prism using non-linear regression fit to normalized data with a variable slope. IC₅₀ values were calculated by GraphPad Prism at 50% inhibition.

ELISA for phospho- and total-HER2 and Correlation with IC₅₀ Values: Protein was harvested from the parental Ba/F3 cell line and each of the Ba/F3 cell lines expressing HER2 mutations as described above. 5 μg/ml of protein was added to each ELISA plate and ELISA was performed as described by the manufacture instructions for phosphorylated HER2 Cell signaling, #7968) and total HER2 (Cell Signaling, #7310). Relative p-HER2 expression was determined by taking the ratio of p-HER2 over total HER2 as determined by ELISA. The relative p-HER2 ratio was plotted against poziotinib IC₅₀ values calculated as described above. Pearson correlations and p-values were determined by GraphPad Prism.

Tyrosine Kinase Inhibitors and T-DM1: All inhibitors were purchased from Selleck Chemical with the exception of EGF816 and pyrotinib which were purchased from MedChem Express. All inhibitors were dissolved in DMSO at a concentration of 10 mM and stored at −80° C. Inhibitors were limited to two freeze thaw/cycle before being discarded. T-DM1 was purchased reconstituted from the M.D. Anderson Cancer Center institutional pharmacy.

Molecular Dynamics Simulations: Protein structural models of the HER2 mutants were constructed using the MOE computer program (Chemical Computing Group) by introducing in silico mutations to the PDB 3PP0 X-ray structure (Aertgeerts et al., 2011). Classical and accelerated molecular dynamics simulations were performed using the NAMD simulation package (Phillips et al., 2005).

Human Cell lines: MCF10A cells were purchased from ATCC and were cultured in DMEM/F12 media supplemented with 1% penicillin/streptomycin, 5% horse serum (sigma), 20 ng/ml EGF, 0.5 mg/ml hydrocortisone, and 10 μg/ml insulin. Stable cell lines were created by retroviral transduction, and retroviruses were generated by transfecting pBabe-Puro based vectors summarized in Table 1 (Addgene and Bioinnovatise) into Phoenix 293T-ampho cells (Orbigen) using Lipofectamine 2000 (Invitrogen). Two days after transduction, 0.5 μg/ml puromycin (Invitrogen) was added to the RPMI media. After 14 days of selection, cells were tested in cell viability assays as described above. CW-2 cells were provided by the Riken cell line database under MTA, and were maintained in RPMI containing 10% FBS and 1% penicillin/streptomycin.

In vivo xenograft studies: CW-2 cell line xenografts were created by injecting 1×10⁶ cells in 50% matrigel into 6 week old female nu/nu nude mice. When tumors reached 350 mm³ mice were randomized into 4 groups: 20 mg/kg afatinib, 5 mg/kg poziotinib, 30 mg/kg neratinib, or vehicle control (0.5% Methylcellulose, 2% Tween-80 in dH2O). Tumor volumes were measured three times per week. Mice received drug Monday-Friday (5 days per week), but began dosing on Wednesday allowing for a 2 day holiday after the first 3 days of dosing.

Y772dupYVMA PDX mice were purchased from Jax Labs (Model #TM01446). Fragments from tumors expressing HER2 Y772dupYVMA were inoculated into 5- to 6-week old female NSG mice (Jax Labs #005557). Mice were measured three times per week, and when tumors reached a volume of 200-300 mm³ mice were randomized into four treatment groups: vehicle control (0.5% Methylcellulose, 0.05% Tween-80 in dH₂O), 2.5 mg/kg poziotinib, 10 mg/kg T-DM1, or combination of 2.5 mg/kg poziotinib and 10 mg/kg T-DM1. Tumor volumes and body weight were measured three times per week. Mice treated with 2.5 mg/kg poziotinib received drug orally Monday-Friday (5 days per week). Mice treated with 10 mg/kg T-DM1 received one intravenous (IV) dose of T-DM1 on the day of randomization. Mice treated with combination poziotinib and T-DM1 received one IV dose of T-DM1 and began 2.5 mg/kg poziotinib five days per week, 3 days after the dose of T-DM1. Mice received a holiday from dosing if the mouse dropped in body weight by greater than 10% or if body weight dropped below 20 grams. Experiments were completed in agreement with Good Animal Practices and with approval from MD Anderson Cancer Center Institutional Animal Care and Use Committee (Houston, Tex.).

FACS: MCF10A cells overexpressing HER2 mutations were plated overnight in a 6-well plate, then treated with 10 nM poziotinib. After 24 hours, cells were washed twice with PBS, and trypsinized. Cells were then resuspended in 0.5% FBS in PBS, and stained with anti-HER2-FITC antibody from Biolegend (#324404) for 45 minutes on ice. Cells were washed with 0.5% FBS in PBS twice, and analyzed by flow cytometry. IgG and unstained controls were used for gating.

Western Blotting: For Western blotting, cells were washed in PBS and lysed in RIPPA lysis buffer (ThermoFisher) and protease inhibitor cocktail tablets (Roche). Protein (30-40 μg) was loaded into gels purchased from BioRad. BioRad semi-dry transfer was used and then probed with antibodies against, pHER2, HER2, pPI3K, PI3K, p-AKT, AKT, p-ERK1/2, and ERK1/2 (1:1000; Cell Signaling). Blots were probed with antibodies against vinculin or β-actin (Sigma-Aldrich) as a loading control, and exposed using ECL Western Blotting substrate (Promega).

HER2 expression level and correlation with Ba/F3 mutant IC50: Protein was harvested from Ba/F cell lines, and ELISAs were performed as described by the manufacture instructions for total HER2 (Cell Signaling, #7310). Relative expression determined by ELISA was plotted against IC50 values calculated as described above. Pearson correlations and p-values were determined by GraphPad Prism.

Clinical Trial and CIND Identifiers: Patients provided written informed consent for treatment with poziotinib on either compassionate use protocol (MD Anderson Cancer Center CIND-18-0014) or clinical trial NCT03066206. The protocols are approved by both the MD Anderson Cancer Center institutional review board and the Food and Drug Administration.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aertgeerts et al., J Biol Chem 2011; 286:18756-65.
Arcila et al., Clin Cancer Res 18:4910-8, 2012.
Arcila et al., Mol Cancer Ther 12(2):220-229, 2013.
Austin-Ward and Villaseca, Revista Medica de Chile, 126(7):838-845, 1998.
Ausubel et al., Current Protocols in Molecular Biology, John Wiley &amp; Sons, New York, N.Y., 2003.
Bose et al., Cancer Discov 2013; 3:224-37.
Bukowski et al., Clinical Cancer Res., 4(10):2337-2347, 1998.
Camacho et al. J Clin Oncology 22(145): Abstract No. 2505 (antibody CP-675206), 2004.
Cha et al. Int J Cancer 130:2445-54, 2012.
Chee et al., Science, 274:610-614, 1996.
Cho et al., Cancer Res 73:6770-9, 2013.
Christodoulides et al., Microbiology, 144(Pt 11):3027-3037, 1998.
Church and Gilbert, Proc. Natl. Acad. Sci. USA 81:1991-1995 (1988).
Cotton et al., Proc. Natl. Acad. Sci. USA 85:4397-4401 (1985).
Davidson et al., J. Immunother., 21(5):389-398, 1998.
Davies et al., Plos One 8, 2013.
Del Tito et al., Clinical Chemistry 44:731-739, 1998.
Drmanac et al., Nat. Biotechnol., 16:54-58, 1998.
Drmanac et al., Science, 260:1649-1652, 1993.
Flavell et al., Cell 15:25 (1978).
Fu et al., Nat. Biotechnol., 16:381-384, 1998/
Geever et al., Proc. Natl. Acad. Sci. USA 78:5081 (1981).
Greulich et al., Proc Natl Acad Sci USA 2012; 109:14476-81.
Hainsworth et al., J Clin Oncol 2018; 36:536-42.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hong et al., J Biol Chem 282:19781-7, 2007.
Hui and Hashimoto, Infection Immun., 66(11):5329-5336, 1998.
Hurwitz et al. Proc Natl Acad Sci USA 95(17): 10067-10071, 1998.
Hyman et al., Nature 2018; 554:189-94.
International Patent Publication No. WO 99/57318
International Patent Publication No. WO1995001994
International Patent Publication No. WO1998042752
International Patent Publication No. WO2000037504
International Patent Publication No. WO2001014424
International Patent Publication No. WO2009/101611
International Patent Publication No. WO2009/114335
International Patent Publication No. WO2010/027827
International Patent Publication No. WO2011/066342
International Patent Publication No. WO2015016718
International Patent Publication No. WO 00/37504
International Patent Publication No. WO01/14424
International Patent Publication No. WO98/42752
Kosaka et al., Cancer Res 2017.
Kosaka et al., Cancer Res 2017.
Kris et al., Ann Oncol 2015; 26:1421-7.
Leal, M., Ann N Y Acad Sci 1321, 41-54, 2014.
Li et al., J Clin Oncol 2018; 36:2532-7.
Lynch et al., N Engl J Med. 350(21):2129-2139, 2004.
Maemondo et al., N Engl J Med 362:2380-8, 2010.
Mazieres et al., J Clin Oncol 2015; 33.
Mitsudomi and Yatabe, Cancer Sci. 98(12):1817-1824, 2007.
Mokyr et al. Cancer Res 58:5301-5304, 1998.
Nagano et al., Clin Cancer Res 2018.
Oxnard et al., J Thorac Oncol. 8(2):179-184, 2013.
Paez et al., Science 304(5676):1497-1500, 2004.
Pao et al., Proc Nall Acad Sci USA 101(36):13306-13311, 2004.
Pardoll, Nat Rev Cancer, 12(4): 252-64, 2012.
Perera et al., Proc Nall Acad Sci USA 106:474-9, 2009.
Phillips et al., J Comput Chem 2005; 26:1781-802.
Qin et al., Proc. Natl. Acad. Sci. USA, 95(24):14411-14416, 1998.
Raca et al., Genet Test 8(4):387-94 (2004).
Robichaux et al., Nat Med 2018; 24:638-46.
Sanger et al., Proc. Natl. Acad. Sci. USA 74:5463-5467 (1977).
Sears et al., Biotechniques, 13:626-633, 1992.
Sheffield et al., Proc. Natl. Acad. Sci. USA 86:232-236 (1989).
Shen et al., J Recept Signal Transduct Res 36:89-97, 2016.
Thress et al., Nat Med 21:560-2, 2015.
U.S. Pat. No. 4,870,287
U.S. Pat. No. 5,288,644
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,869,245
U.S. Pat. No. 5,885,796
U.S. Pat. No. 6,207,156
U.S. Pat. No. 8,008,449
U.S. Pat. No. 8,017,114
U.S. Pat. No. 8,119,129
U.S. Pat. No. 8,188,102
U.S. Pat. No. 8,329,867
U.S. Pat. No. 8,354,509
U.S. Pat. No. 8,735,553
U.S. Patent Publication No. 2004/0014095
U.S. Patent Publication No. 2005/0260186
U.S. Patent Publication No. 2006/0104968
U.S. Patent Publication No. 20110008369
U.S. Patent Publication No. 20130071452
U.S. Patent Publication No. 2014022021
U.S. Patent Publication No. 20140294898
Underhill et al., Genome Res. 7:996-1005 (1997).
Yang et al., Int J Cancer 2016.
Yasuda et al., Sci Transl Med 5(216):216ra177, 2013.
Zimmerman et al., Methods Mol. Cell. Biol., 3:39-42, 1992.

What is claimed is:

1. A method of treating a cancer in a subject comprising administering an effective amount of a tyrosine kinase inhibitor (TKI) and a HER antibody-drug conjugate to the subject.

2. The method of claim 1, wherein the HER2 antibody is trastuzumab.

3. The method of claim 1, wherein the HER2 antibody-drug conjugate is trastuzumab emtansine (T-DM1).

4. The method of any of claims 1-3, wherein the TKI is a Quinazolinamine-based TKI.

5. The method of claim 4, wherein the Quinazolinamine-based TKI is poziotinib, afatinib, neratinib, dacomitinib, or tarloxotinib.

6. The method of claim 4, wherein the Quinazolinamine-based TKI is poziotinib.

7. The method of claim 6, wherein the poziotinib is administered at a dose of less than 16 mg.

8. The method of any of claims 1-7, wherein the subject is administered poziotinib and T-DM1.

9. The method of claim 8, wherein the subject is administered a single dose of T-DM1.

10. The method of any of claims 1-9, wherein the TKI is administered prior to the HER2 antibody-drug conjugate.

11. The method of any of claims 1-9, wherein the TKI is administered after the HER2 antibody-drug conjugate.

12. The method of any of claims 1-9, wherein the TKI is administered simultaneously with the HER2 antibody-drug conjugate.

13. The method of any of claims 1-12, wherein the cancer is a HER2 mutant cancer.

14. The method of claim 13, wherein the HER2 mutant cancer comprises activating mutations of HER2 within the tyrosine kinase domains spanning exons 19-21.

15. The method of claim 13, wherein the HER2 mutant cancer comprises one or more mutations selected from the group consisting of V754M, L755S, L755P, D769H, D769N, D769Y, V773M, V777L, Y772dupYVMA, G776delinsVC, G776delinsVV, G776delinsLC, V777insCG, G778insLPS, P780insGSP, L786V, V842I, and L869R.

16. The method of claim 13, wherein the HER2 mutant cancer has an exon 19, exon 20, and/or exon 21 mutation.

17. The method of claim 16, wherein the HER2 mutant cancer has an exon 20 mutation.

18. The method of claim 17, wherein the exon 20 mutation comprises one or more point mutations, insertions, and/or deletions of 1-18 nucleotides between amino acids E770-R786 of HER2.

19. The method of claim 18, wherein the exon 20 mutation is at residue Y772, V773, A775, G776, V777, G778, 5779, and/or P780.

20. The method of claim 17, wherein the exon 20 mutation is an exon 20 insertion mutation.

21. The method of claim 20, wherein the exon 20 insertion mutation is Y772dupYVMA, G778dupGSP, and/or G776delinsVC.

22. The method of claim 16, wherein the exon 19 mutation is at residue L755 or D769.

23. The method of claim 16, wherein the exon 19 mutation is L755P.

24. The method of claim 16, wherein the exon 20 mutation is a point mutation.

25. The method of claim 24, wherein the exon 20 point mutation is at residue C805.

26. The method of claim 25, wherein the exon 20 point mutation is C805S.

27. The method of claim 16, wherein the exon 21 mutation is a point mutation.

28. The method of claim 27, wherein the point mutation is at residue V842 or L869.

29. The method of claim 28, wherein the point mutation is V842I or L869R.

30. The method of any of claims 1-29, wherein the cancer is lung cancer.

31. The method of claim 30, wherein the lung cancer is non-small cell lung cancer (NSCLC).

32. The method of claim 6, wherein the poziotinib is administered orally.

33. The method of claim 32, wherein the poziotinib is administered at a dose of 5-25 mg.

34. The method of claim 32, wherein the poziotinib is administered at a dose of 8 mg, 12 mg, or 16 mg.

35. The method of claim 34, wherein the poziotinib is further defined as poziotinib hydrochloride salt.

36. The method of claim 35, wherein the poziotinib hydrochloride salt is formulated as a tablet.

37. The method of any of claims 6-36, wherein the poziotinib and/or the T-DM1 are administered intravenously, subcutaneously, intraosseously, orally, transdermally, in sustained release, in controlled release, in delayed release, as a suppository, or sublingually.

38. The method of any of claims 1-37, further comprising administering an additional anti-cancer therapy.

39. The method of claim 38, wherein the additional anti-cancer therapy is chemotherapy, radiotherapy, gene therapy, surgery, hormonal therapy, anti-angiogenic therapy or immunotherapy.

40. The method of any of claims 1-39, wherein the subject is human.

41. A pharmaceutical composition comprising a TKI and a HER antibody-drug conjugate.

42. The composition of claim 41, wherein the TKI is poziotinib and the HER antibody-drug conjugate is T-DM1.

43. A method of predicting a response to a TKI in combination with a HER2 antibody-drug conjugate in a subject having a cancer comprising detecting a HER2 mutation in a genomic sample obtained from said subject, wherein if the sample is positive for the presence of the HER2 mutation, then the patient is predicted to have a favorable response to the poziotinib in combination with the HER2 antibody-drug conjugate an anti-cancer therapy.

44. The method of claim 43, wherein the HER2 mutation is an exon 19, exon 20, and/or exon 21 mutation.

45. The method of claim 44, wherein the exon 20 mutation comprises one or more point mutations, insertions, and/or deletions of 1-18 nucleotides between amino acids E770-R786 of HER2.

46. The method of claim 44, wherein the exon 20 mutation is at residue Y772, V773, A775, G776, V777, G778, 5779, and/or P780.

47. The method of claim 45, wherein the exon 20 insertion mutation is Y772dupYVMA, G778dupGSP, and/or G776delinsVC.

48. The method of claim 44, wherein the exon 19 mutation is at residue L755 or D769.

49. The method of claim 44, wherein the exon 20 mutation is at residue C805.

50. The method of any of claims 43-49, wherein the genomic sample is isolated from saliva, blood, urine, normal tissue, or tumor tissue.

51. The method of any of claims 43-50, wherein the presence of a HER2 mutation is determined by nucleic acid sequencing or PCR analyses.

52. The method of any of claims 43-51, wherein a favorable response to the TKI in combination with the HER antibody-drug conjugate comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, or increased patient survival.

53. The method of any of claims 43-53, wherein the TKI is poziotinib and the HER antibody-drug conjugate is T-DM1.

54. The method of any of claims 43-53, further comprising administering the TKI in combination with the HER2 antibody-drug conjugate to said subject predicted to have a favorable response.

55. The method of any of claims 43-54, further comprising administering poziotinib in combination with T-DM1 to said subject predicted to have a favorable response.


SEQUENCE LISTING (1 sequence)

SEQ ID NO: 1 - DNA, 12 bp, Artificial sequence
  Synthetic oligonucleotide
tatgtcatggct